SEQUENCE LISTING (10 sequences)

SEQ ID NO: 1 - protein, 473 aa, Bacillus subtilis
MNIKKFAKQATVLTFTTALLAGGATQAFAKETNQKPYKETYGISHITRHDMLQIPEQQKNEKYQVPEFDPSTIKNISSAKGLDVWDSWPLQNADGTVANYHGYHIVFALAGDPKNADDTSIYMFYQKVGETSIDSWKNAGRVFKDSDKFDANDSILKDQTQEWSGSATFTSDGKIRLFYTDFSGKHYGKQTLTTAQVNVSASNSSLNINGVEDYKSIFDGDGKTYQNVQQFIDEGNYSSGDNHTLRDPHYVEDKGHKYLVFEANTGTEDGYQGEESLFNKAYYGKSTSFFRQESQKLLQSDKKRTAELANGALGMIELNDDYTLKKVMKPLIASNTVTDEIERANVFKMNGKWYLFTDSRGSKMTIDGITSNDIYMLGYVSNSLTGPYKPLNKTGLVLKMDLDPNDVTFTYSHFAVPQAKGNNVVITSYMTNRGFYADKQSTFAPSFLLNIKGKKTSVVKDSILEQGQLTVNK

SEQ ID NO: 2 - DNA, 1422 bp, Bacillus subtilis
  source 1..1422: mol_type = genomic DNA
atgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataa

SEQ ID NO: 3 - DNA, 1038 bp, unidentified
  source 1..1038: mol_type = genomic DNA
atgaccattcattcctctgcgctcagtgcaatcggcaacacgccgctcatccgtctgaaggctgcctctgaagcgacggggtgtgaaatcctcggcaaggcggaattcctcaatcccggccagtcggtgaaggatcgtgcagcgctctacatcatccgcgacgccgaacggcgcggccaattgcgcccgggcggcaccatcgtggaaggcacggccggaaataccggcatcggcctgtcgctggtcgccaatgcgctcggctacaagacggtgatcgtcattcccgagacgcagagccaggagaagaaggacgcgctgaagcttctcggcgccaagctggtcgaagtaccggccgcgccttattccaatccgaacaattacgtaaaggtctccggccggcttgcaaaacagctcgccgccacggatgaaaacggcgcgatctgggccaaccagttcgacaatatcgccaaccgtcaggcgcatatcgagacgaccgcgcctgagatctgggaccagacggacggcaaggtcgatggcttcatatgctcggtcggttccggcggaacactggccggcgtcgccgacggcctgcgcgatttcaatcccgacatcaagatcggtcttgccgaccctgaaggtgccgccctttatgaattttacaaacacggcgtcctgaagtccgaaggctcctccattaccgaaggcatcggacaggggcgcatcaccgccaatctggaagggttcgcaccggatttctcctatcgtatttcggacgcggaagcgctgccggtgctgttcgatctggtcacgaaagaaggcctgtgcctcggcggttcttccggcatcaacattgccggcgcaatccgccttgcccgtgagctcggtccggggcatacaatcgtgacgatactgtgcgactacggtaatcgttatcagtcgaagctcttcaatccggatttcctgcgctccaaggggcttcccgtgccgcagtggctggcgaccaagagcgagattccggttccctacgaaaccgtttga

SEQ ID NO: 4 - protein, 345 aa, unidentified
MTIHSSALSAIGNTPLIRLKAASEATGCEILGKAEFLNPGQSVKDRAALYIIRDAERRGQLRPGGTIVEGTAGNTGIGLSLVANALGYKTVIVIPETQSQEKKDALKLLGAKLVEVPAAPYSNPNNYVKVSGRLAKQLAATDENGAIWANQFDNIANRQAHIETTAPEIWDQTDGKVDGFICSVGSGGTLAGVADGLRDFNPDIKIGLADPEGAALYEFYKHGVLKSEGSSITEGIGQGRITANLEGFAPDFSYRISDAEALPVLFDLVTKEGLCLGGSSGINIAGAIRLARELGPGHTIVTILCDYGNRYQSKLFNPDFLRSKGLPVPQWLATKSEIPVPYETV

SEQ ID NO: 5 - DNA, 1062 bp, Escherichia coli
  source 1..1062: mol_type = genomic DNA
atggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgataacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcaccgtttaaacaggctgaatttcagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcaactgcctggctgaaagataacccggaaaccgcgaaagagatcgagaagaaagtacgtgagttgctgctgagcaacccgaactcaacgccggatttctctgtagatgacagcgaagacgtagcagaaactaacgaagatttttaa

SEQ ID NO: 6 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEDVAETNEDF

SEQ ID NO: 7 - DNA, 969 bp, unidentified
  source 1..969: mol_type = genomic DNA
atgcccgaagccagaaagcccggacgcggacgcgtctattcctcaatcacggaaacaatcggcgacacgccgatcgtgaggctcgacaagctggcgaaggaaaagggcgtgaaggcaaacctgctcgccaagctggagttcttcaaccctatcggttccgtgaaggaccgtatcggcgttgccatgatcgaagcgctggaagcacagggtaagatcactcccggcaagacgacgctggtggagccgacttcaggtaataccggcatcgcgctcgcctttgccgccgccgccaagggttacaagctgatcctcaccatgccggaaaccatgtcggttgaacgcagaaagatgctggcgctgctgggtgccgagctggtgctgaccgaaggcccgaagggcatgaagggcgccattgccaaggctcaggaactggcagagaccctgcccgacgccgtcattccccaacaattcgaaaaccccgccaatccggatattcaccgcaggacaacggcggaagaaatctggaacgacaccgatggcacggtcgatatcctcgtttccgggatcggcaccggcggcacgatcaccggcacgggccaggttctgaaatcccgcaagcccgaagtgaaggtgattgcggtcgagcccgccgacagcccggttctttccggcggcaatcccggcccgcacaagattcagggcatcggcgccggctttgcacccgccattctcgacaccggcgtctatgatgaaatcgtcaccgtgaccaatgacgaggcatttgaaattgcgcgtctggcggcacggctcgaaggcgtgccggtcggcatctcctcgggtgcggcactggccgcggccatcaaggtcggcgcgcgtgaggaaaatgccggcaagaacatcgtcgtcatcattccttctttcgcagaacgttatctttcgaccgcgcttttcgaaggccttgggctttaa

SEQ ID NO: 8 - protein, 322 aa, unidentified
MPEARKPGRGRVYSSITETIGDTPIVRLDKLAKEKGVKANLLAKLEFFNPIGSVKDRIGVAMIEALEAQGKITPGKTTLVEPTSGNTGIALAFAAAAKGYKLILTMPETMSVERRKMLALLGAELVLTEGPKGMKGAIAKAQELAETLPDAVIPQQFENPANPDIHRRTTAEEIWNDTDGTVDILVSGIGTGGTITGTGQVLKSRKPEVKVIAVEPADSPVLSGGNPGPHKIQGIGAGFAPAILDTGVYDEIVTVTNDEAFEIARLAARLEGVPVGISSGAALAAAIKVGAREENAGKNIVVIIPSFAERYLSTALFEGLGL

SEQ ID NO: 9 - DNA, 1038 bp, unidentified
  source 1..1038: mol_type = genomic DNA
atgaccattcactcctctgcgctcagtgccatcggcaatacgccgctcatccgtctgaaagccgcctctgaagcgacggggtgtgaaatcctcggcaaggcggagttcctcaatcccggccagtcggtgaaggatcgcgccgcgctctacatcatccgcgacgccgaacggcgcgggcagttgcggccgggtggcactattgtggaaggcacggcgggcaataccggcatcggcctgtcgctggtcgccaatgcgctcggctacaagacggtcatcgtcattcccgagacgcagagccaggaaaagaaggacgcgctgaaacttcttggcgcgaagctggtcgaggttccggcagccccctattccaacccgaacaattacgtaaaggtctccgggcggctggcaaaacagcttgccgcgaccgacgagaacggcgcgatctgggccaaccagttcgacaatatcgccaaccgtcaggcccatatcgaaacgaccgctcctgaaatctgggaccagacggatggcaaggtcgatggtttcatatgctcggtcggttccggcggaacacttgcgggcgttgccgatggcctgcgcgatttcaatcctgacatcaagatcggccttgccgatcccgaaggcgccgccctttacgaattttacaagcacggcgtgctgaagtcggaaggttcctccatcaccgagggcatcggacaggggcgcatcaccgccaatctggaaggtttcacgccggatttctcctatcgcatttcggatgcggaagccctgccggtgctgttcgatctggtcaccaaagagggtctgtgcctcggcggctcctccggcatcaacattgccggcgcaatccgccttgcccgcgagctcggtccggggcatacaatcgtgacgatattgtgcgactacggcaatcgttaccagtcgaagctcttcaatccggatttcctgcgttccaagggacttccggttccacagtggctggcgacgaaaagcgagattgcggttccgtacgaaaccgtttga

SEQ ID NO: 10 - protein, 345 aa, unidentified
MTIHSSALSAIGNTPLIRLKAASEATGCEILGKAEFLNPGQSVKDRAALYIIRDAERRGQLRPGGTIVEGTAGNTGIGLSLVANALGYKTVIVIPETQSQEKKDALKLLGAKLVEVPAAPYSNPNNYVKVSGRLAKQLAATDENGAIWANQFDNIANRQAHIETTAPEIWDQTDGKVDGFICSVGSGGTLAGVADGLRDFNPDIKIGLADPEGAALYEFYKHGVLKSEGSSITEGIGQGRITANLEGFTPDFSYRISDAEALPVLFDLVTKEGLCLGGSSGINIAGAIRLARELGPGHTIVTILCDYGNRYQSKLFNPDFLRSKGLPVPQWLATKSEIAVPYETV

(19) United States
(12) Patent Application Publication
(10) Pub. No.: US 2024/0150776 A1
(43) Pub. Date: May 9, 2024

(54) NOVEL AGROBACTERIUM WITH NO OVERGROWTH IN PLANT TRANSFORMATION

(71) Applicant: Benson Hill, Inc., St. Louis, MO (US)

(72) Inventors: Shujun Chang, Wildwood, MO (US); Sunilkumar Ganesan, Wildwood, MO (US); Fred Moshiri, Chesterfield, MO (US)

(21) Appl. No.: 18/499,789

(22) Filed: Nov. 1, 2023

Related U.S. Application Data
(60) Provisional application No. 63/382,166, filed on Nov. 3, 2022.

(51) Int. Cl. C12N 15/82; C12N 1/20; C12N 9/10; C12N 15/74

(52) U.S. Cl. CPC C12N 15/8205; C12N 1/20; C12N 9/1055; C12N 9/1085; C12N 15/743; C12Y 204/0101; C12Y 205/01047; C12N 2800/101

(57) ABSTRACT

The instant disclosure provides a genetically modified bacterial cell, i.e., modified Agrobacterium strain, which expresses a levansucrase and has reduced expression of endogenous CysK. As a result, the growth of the modified bacterial cell on medium or tissue culture with sucrose and deficient in cysteine is inhibited. The two-action growth control of the modified Agrobacterium strain can overcome the unmet issue of Agrobacterium overgrowth during plant transformation, and therefore increase the efficiency of plant transformation.

### CROSS-REFERENCE TO RELATED APPLICATIONS

This application claims the benefit of U.S. Provisional Application No. 63/382,166, filed Nov. 3, 2022, which is incorporated by reference herein in its entirety.

### REFERENCE TO A SEQUENCE LISTING SUBMITTED AS AN XML FILE VIA PATENT CENTER

The application contains a sequence listing which is submitted herewith in electronically readable format. The Sequence Listing file was created on Nov. 1, 2023, is named B88552_1390US.1_0360.1_Seq_List.xml and its size is 17,004 bytes. The entire contents of the Sequence Listing are hereby incorporated by reference herein.

### FIELD OF THE INVENTION

The present disclosure relates to compositions and methods for modifying Agrobacterium to reduce overgrowth during plant transformation such that transformation efficiency is increased when compared to transforming a plant or plant part with a control cell.

### BACKGROUND OF THE INVENTION

Agrobacterium overgrowth is a common issue in Agrobacterium-mediated plant transformation. It happens very often after transformation even in the presence of antibiotics such as Timentin, carbenicillin, and cefotaxime as the bacteria can develop resistance to these antibiotics or the antibiotics break down under tissue culture conditions. The consequences of the overgrowth include lower transformation efficiency, poor plant health and spending more resources to manage and clean up the contaminated culture. Additional challenges include that certainly strains, such as AGL1, have carbenicillin resistant gene. Thus, researchers can't use carbenicillin to control such strains on selection and regeneration media.

In recent years two strategies have been developed to modify the Agrobacterium to limit the Agrobacterium overgrowth issue. One strategy is to create mutant Agrobacterium that is auxotrophic to certain amino acids. One example is the thymidine auxotrophic Agrobacterium strain LBA4404 (U.S. Pat. No. 8,334,429). Thymidine auxotrophic Agrobacterium C58 strain or derivatives such as EHA101 and 105 have been developed. Moreover, adenine, leucine and cysteine auxotrophic Agrobacterium strains have also been developed. Another strategy is to insert SacB-SacR genes from Bacillus subtilis as a negative selection on sucrose rich medium. SacB-SacR genes encode levansucrase that converts sucrose to levan, which is toxic to gram negative bacteria such as Agro. Both strategies have been demonstrated functional in plant transformation, but with the limitation that the effectiveness is partial, e.g., Agro can still grow on plant growth medium at about 30% of explants in the case of infection with mutant strain carrying SacB-SacR genes. Thus new strategies for preventing Agrobacterium overgrowth during plant transformation would be helpful in the art.

### SUMMARY OF THE INVENTION

The present disclosure presents a modified bacterial cell comprising a heterologous polynucleotide comprising a nucleic acid sequence encoding a levansucrase enzyme. In some embodiments, an endogenous CysK gene has reduced expression when compared to a proper control bacterium. In some specific embodiments, an endogenous CysK gene expression is reduced due to a mutation in the CysK gene or regulatory region of the CysK gene.

In some embodiments, the mutation is an insertion. In some embodiments, the insertion is an insertion of a heterologous polynucleotide comprising a nucleic acid sequence encoding the levansucrase enzyme.

In some embodiments, the nucleic acid molecule encoding the levansucrase enzyme is a SacB/SacR gene. In some specific embodiments, the SacB/SacR gene comprises (i) at least 80%, or at least 85%, or at least 90%, or at least 95%, or at least 99% sequence identity with SEQ ID NO: 2, and retains levansucrase activity; or (ii) the nucleotide sequence of SEQ ID NO: 2. In certain embodiments, the levansucrase enzyme comprises (i) at least 80%, or at least 85%, or at least 90%, or at least 95%, or at least 99% amino acid sequence identity with SEQ ID NO: 1, and retains levansucrase activity; or (ii) the amino acid sequence of SEQ ID NO: 1.

In some embodiments, the endogenous CysK coding sequence comprises (i) at least 80%, or at least 85%, or at least 90%, or at least 95%, or at least 99% sequence identity with any one of SEQ ID NO: 3, SEQ ID NO: 7, or SEQ ID NO: 9, and retains CysK activity; or (ii) the nucleotide sequence of SEQ ID NO: 3, SEQ ID NO: 7, or SEQ ID NO: 9. In some specific embodiments, the endogenous CysK protein comprises (i) at least 80%, or at least 85%, or at least 90%, or at least 95%, or at least 99% sequence identity with any one of SEQ ID NO: 4, SEQ ID NO: 8 or SEQ ID NO: 10, and retains CysK activity; or (ii) the amino acid sequence of SEQ ID NO: 4, SEQ ID NO: 8 or SEQ ID NO: 10.

In some embodiments, the endogenous level or activity of CysK is reduced by at least 20%, 30%, 40%, 50%, 60%, 70%, 80%, 90%, 95%, 99%, or 100%.

In some embodiments, the modified bacterial cell is cysteine auxotrophic. In some embodiments, the modified bacterial cell is a Gram-negative bacterium. In some embodiments, the modified bacterial cell is an Agrobacterium. In some further embodiments, the modified bacterial cell further comprises one or more heterologous polynucleotide of interest. In one specific embodiment, the one or more heterologous polynucleotide of interest encodes an editing reagent and a repair template, and wherein homology-directed repair (HDR) is increased, an HDR to non-homologous end-joining (NHEJ) ratio is increased, chimerism is reduced, mosaicism is reduced, and/or uniform editing of a plant genome is increased relative to a control plant or plant part, when the DNA construct is introduced in a plant or plant part.

In some embodiments, the growth of the cell in a medium deficient in cysteine is inhibited. In some embodiments, the growth of the modified bacterial cell in a plant tissue culture medium deficient in cysteine is inhibited. In some embodiments, the growth of the modified bacterial cell in a medium comprising sucrose is inhibited. In yet some embodiments, the growth of the modified bacterial cell in a plant tissue culture medium comprising sucrose is inhibited. In certain embodiments, the growth of the modified bacterial cell in a plant tissue culture medium comprising sucrose and deficient in cysteine is inhibited when compared to a control cell. In some specific embodiments, the growth of the modified bacterial cell in a plant tissue culture medium comprising sucrose and deficient in cysteine is inhibited by at least about 20%, 30%, 40%, 50%, 60%, 70%, 80%, 90%, 95%, 99%, or 100%.

The present disclosure further provides a population of the modified bacterial cell described herein. In some embodiments, the population is heterogeneous. In some embodiments, about 75%, about 80%, about 85%, about 90%, about 95%, or about 99% cells of the population contain the introduced polynucleotide; and/or wherein about 75%, about 80%, about 85%, about 90%, about 95%, or about 99% cells of the population express a levansucrase and have reduced endogenous cysK expression.

In some embodiments, the population is homogenous, or wherein all cells of the population express a levansucrase and have reduced endogenous cysK expression. In some embodiments, the population comprises reduced levels or activity of CysK and/or increased levels or activity of levansucrase when compared to a population of control cells.

In one specific embodiment, the levansucrase is encoded by a SacB/SacR gene.

The present disclosure further provides a polynucleotide comprising from the 5′ to 3′: (a) a 5′ fragment of a CysK coding sequence; (b) a levansucrase coding sequence; and (c) a 3′ fragment of the CysK coding sequence.

In some embodiments, the nucleic acid molecule encoding the levansucrase enzyme is a SacB/SacR gene. In some further embodiments, the SacB/SacR gene comprises (i) at least 80%, or at least 85%, or at least 90%, or at least 95%, or at least 99% sequence identity with SEQ ID NO: 2, and retains levansucrase activity; or (ii) the polynucleotide sequence of SEQ ID NO: 2. In some embodiments, the levansucrase comprises (i) at least 80%, or at least 85%, or at least 90%, or at least 95%, or at least 99% sequence identity with SEQ ID NO: 1, and retains levansucrase activity; or (ii) the amino acid sequence SEQ ID NO: 1.

In some embodiments, the CysK coding sequence comprises (i) at least 80%, or at least 85%, or at least 90%, or at least 95%, or at least 99% sequence identity with any one of SEQ ID NO: 3, SEQ ID NO: 7 or SEQ ID NO: 9, and retains CysK activity; or (ii) the polynucleotide sequence of SEQ ID NO: 3, SEQ ID NO: 7 or SEQ ID NO: 9. In some embodiments, the CysK comprises (i) at least 80%, or at least 85%, or at least 90%, or at least 95%, or at least 99% sequence identity with any one of SEQ ID NO: 4, SEQ ID NO: 8 or SEQ ID NO: 10, and retains CysK activity; or (ii) the amino acid sequence of SEQ ID NO: 4, SEQ ID NO: 8 or SEQ ID NO: 10.

In some embodiments, the polynucleotide further comprises a RecA gene. In some embodiments, the polynucleotide further comprises a promoter active in a bacterial cell. In one specific embodiment, the polynucleotide wherein the polynucleotide is a pUC plasmid.

The present disclosure further provides a method of transforming a cell, comprising the steps of: (a) introducing the any one of the polynucleotide described herein into a cell; (b) culturing the cell; (c) selecting for the cells from step (b) which express levansucrase and have reduced expression of CysK.

In some embodiments, the SacB/SacR gene is inserted into the endogenous CysK coding sequence. In some embodiments, insertion of the SacB/SacR gene into the endogenous CysK coding sequence results the expression of the SacB/SacR gene. In some embodiments, the insertion of the SacB/SacR gene reduces the expression of the endogenous CysK gene. In some embodiments, the method further comprises introducing at least one heterologous polynucleotide of interest into the cell in step (a).

In some embodiments, the at least one heterologous polynucleotide of interest encodes an editing reagent and a repair template, and wherein homology-directed repair (HDR) is increased, an HDR to non-homologous end-joining (NHEJ) ratio is increased, chimerism is reduced, mosaicism is reduced, and/or uniform editing of a plant genome is increased relative to a control plant or plant part, when the DNA construct is introduced in a plant or plant part.

In some embodiments, the expression of levansucrase or CysK is measured by western blot or ELISA.

In some embodiments, the cell is a Gram-negative bacterium. In some embodiments, the cell is an Agrobacterium.

The present disclosure further provides a method of transforming a plant or plant part using the cell or the population of cells described herein. In some embodiments, the transformation efficiency is increased when compared to transforming a plant or plant part with a control cell. In some embodiments, the transformation efficiency is increased by at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000%.

In some embodiments, the plant is an explant or a seed. In some embodiments, the plant is any one of corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, conifers, legume, Fabaceae (or Leguminosae), beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.), oilseed plant (e.g., canola (Brassica napus), cotton (Gossypium spp.), camelina (Camelina sativa) and sunflower (Helianthus spp.)), wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa).

In one specific embodiment, the plant is a soybean plant.

### BRIEF DESCRIPTION OF THE DRAWINGS

FIG. 1 is a diagram depicting the conversion of sucrose into levan, which is toxic to Gram-negative bacterium, by levansucrase.

FIG. 2 is a diagram depicting the recombination process in which a SacB/SacR is inserted to the Agrobacterium C58 CysK gene loci.

FIG. 3 is a diagram depicting a vector—pLVC18—for use in the recombination process depicted in FIG. 2.

### DETAILED DESCRIPTION

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

### I. Definitions

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, all polynucleotide sequences written using the nucleic acid standard notation of the International Union of Pure and Applied Chemistry (IUPAC, Biochemistry (1970) Vol. 9:4022-4027); adenine (A), thymine (T), guanine (G), and cytosine (C) are equivalent to the corresponding RNA polynucleotide sequences. Therefore, “T” (Thymine) in all sequences is equivalent to “U” (uracil). For example, the sequence AATAAA in a DNA coding strand would also indicate the corresponding mRNA sequence AAUAAA.

As used herein, the use of the term “polynucleotide”, “DNA”, or “nucleic acid” is not intended to limit the present invention to polynucleotides comprising DNA. Those of ordinary skill in the art will recognize that polynucleotides can include ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

The instant disclosure encompasses isolated or substantially purified polynucleotide or nucleic acid compositions. An “isolated” or “purified” polynucleotide, or biologically active portion thereof, is substantially or essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived.

As used herein, the term “heterologous” or “exogenous” in reference to a nucleotide sequence or amino acid sequence is intended to mean a sequence that is purely synthetic, that originates from a foreign species, or, if from the same species, is substantially modified from its native form in composition and/or genomic locus by deliberate human intervention.

As used herein, the term “endogenous” in reference to a nucleotide sequence or protein is intended to mean a sequence or protein that is naturally comprised within or expressed by a cell.

As used herein, the term “wild-type” refers to the most common naturally occurring allele (i.e., polynucleotide sequence) in the allele population of the same type of gene; a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which include one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

An auxotrophic bacterium is a mutant bacterium that requires a specific nutrient that is not required by the parent bacterium. Thus, as used herein, a “cysteine auxotrophic” cell, refers to a cell of which the growth is inhibited without supplemental cysteine added to the growth medium. In specific embodiments, the cysteine auxotrophic cell (e.g., bacterial cell) has reduced growth under a cysteine deficient condition. In some embodiments, the cysteine content of a cysteine deficient condition is reduced by about 10%, about 20%, about 30%, about 40%, about 50%, about 60%, about 70%, about 80%, about 90%, about 95%, about 99%, or about 100% when compared to a normal growth medium. Accordingly, the growth of a cysteine auxotrophic cell on a cysteine deficient condition is inhibited by at least about 5%, about 10%, about 20%, about 30%, about 40%, about 50%, about 60%, about 70%, about 80%, about 90%, about 95%, about 99%, or about 100%.

Bacteria can synthesize cysteine from serine via a two-step pathway beginning with the O-acetylation of serine, followed by O-replacement of the acetyl group by sulfide or thiosulfate. The mutation and inactivation of the key enzyme involved in these mechanisms, for example, CysK, can result in a cysteine auxotrophic bacterial cell.

A “plant” refers to a whole plant, any part thereof, or a cell or tissue culture derived from a plant, comprising any of: whole plants, plant components or organs (e.g., leaves, stems, roots, embryos, pollen, ovules, seeds, grains, leaves, flowers, branches, fruit, pulp, juice, kernels, ears, cobs, husks, stalks, root tips, anthers, etc.), plant tissues, plant cell tissue cultures from which plants can be regenerated, plant calli, plant clumps, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants comprising the introduced polynucleotides are also within the scope of the invention. Further provided is a processed plant product (e.g., extract) or byproduct that retains one or more polynucleotides disclosed herein.

Commonly known plant species includes, without limitations, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. Aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of dicots, e.g., legumes.

Plant cells possess nuclear, plastid, and mitochondrial genomes. The polynucleotides and methods of the present invention may be used to modify the sequence of the nuclear, plastid, and/or mitochondrial genome, or may be used to modulate the expression of a gene or genes encoded by the nuclear, plastid, and/or mitochondrial genome. Accordingly, by “chromosome” or “chromosomal” is intended the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct has an artificial combination of nucleic acid fragments, which includes, without limitations, regulatory and coding sequences that are not found together in nature. For example, a recombinant DNA construct may contain regulatory sequences and coding sequences that are derived from different sources, or regulatory sequences and coding sequences derived from the same source and arranged in a manner different than that found in nature. Such a construct may be used by itself or may be used in conjunction with a vector. In some embodiments, a recombinant DNA construct is a plasmid.

As used herein, the term “vector” or “recombinant DNA vector” may be a construct that includes a replication system and sequences that are capable of transcription and translation of a polypeptide-encoding sequence in a given host cell. A “recombination vector” as used herein, refers to a vector which can engage in homologous recombination with the chromosomal DNA of a host cell. If a vector is used, then the choice of vector is dependent upon the method that will be used to transform host cells as is well known to those skilled in the art. Vectors can include, without limitation, plasmid vectors, such as pUC plasmid, or any other vector known in the art suitable for delivering a gene to a target cell. The skilled artisan is well aware of the genetic elements that must be present on the vector in order to successfully transform, select and propagate host cells containing any of the isolated nucleotides or nucleic acid sequences of the invention.

As used herein, the term “inactivate a gene” refers to introducing mutations to a gene such that the function of the gene is lost. The mutations include, without limitations, insertion, deletion, and/or substitution. In some embodiments, the mutations may cause frameshift mutation of the gene, premature stop of translation, and reduced level of the protein encoded by the gene. The expression and/or protein level of an “inactivated gene” may be reduced by at least 5%, 10%, 20%, 30%, 40%, 50%, 60%, 70%, 80%, 90%, 95%, 99%, or 100%. In other embodiments, the mutations do not cause significant change to the level of the protein encoded by the gene. The mutations cause the activity of the protein to be reduced by at least 5%, 10%, 20%, 30%, 40%, 50%, 60%, 70%, 80%, 90%, 95%, 99%, or 100%. In a specific case, endogenous CysK gene is inactivated in a cell.

As used herein, the term “Gram-negative bacterium” or “Gram-negative bacteria” refers to bacteria that do not retain the crystal violet stain used in the Gram staining method of bacterial differentiation (Salton et al., Medical Microbiology, 4th ed.). Gram-negative bacteria are characterized by the cell envelopes, which are composed of a thin peptidoglycan cell wall sandwiched between an inner cytoplasmic cell membrane and a bacterial outer membrane.

As used herein, “Agrobacterium” refers to a genus of Gram-negative bacteria, among which Agrobacterium tumefaciens is the most commonly studied species. Agrobacterium is well known for its ability to transfer DNA between itself and plants, and for this reason it has become an important tool for genetic engineering. The ability of Agrobacterium to transfer genes to plants and fungi is used in biotechnology, for example, for genetic engineering for plant improvement. Genomes of plants and fungi can be engineered by using Agrobacterium for the delivery of sequences hosted in T-DNA binary vectors. A modified Ti or Ri plasmid can be used (Schell et al., 1979, Genetic Engineering for Nitrogen Fixation, 9. pp. 159-79). Agrobacterium can be used to transform various plants, including, without limitations, soybean, cotton, maize, sugar beet, alfalfa, wheat, rapeseed oil (canola), creeping bentgrass and rice. The genes to be introduced into the plant are cloned into a plant binary vector that contains the T-DNA region of the disarmed plasmid, together with a selectable marker (such as antibiotic resistance) to enable selection for plants that have been successfully transformed. Plants are grown on media containing antibiotic following transformation, and those that do not have the T-DNA integrated into their genome will die (Thomson, “Genetic Engineering of Plants”, 2017, Biotechnology, 3).

As used herein, the term “operably linked”, as used herein, is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two polypeptide coding regions, by operably linked is intended that the coding regions are in the same reading frame. The cassette may additionally contain at least one additional gene to be co-transformed into the organism. Alternatively, the additional gene(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain selectable marker genes.

The term “control cell” used herein is a counterpart bacterial cell without the two-action selection mechanism. For example, in some embodiments, a control cell can be a wild-type bacterial cell. In other embodiments, a control cell expresses a levansucrase but have wild-type endogenous CysK gene such that the CysK expression is not reduced. In another embodiment, the control cell has reduced CysK expression but does not express a levansucrase.

The term “homologous recombination”, as used herein, refers to is a type of genetic recombination in which genetic information is exchanged between two similar or identical molecules of double-stranded or single-stranded nucleic acids.

### II. Overview of the Invention

Agrobacterium overgrowth is a common issue in Agrobacterium-mediated plant transformation. It happens very often after transformation even in the presence of antibiotics such as Timentin, carbenicillin, and cefotaxime as the bacteria can develop resistance to these antibiotics or the antibiotics break down under tissue culture conditions. The consequences of the overgrowth include lower transformation efficiency, poor plant health and spending more resources to manage and clean up the contaminated culture.

The instant disclosure is based on a two mode-of-actions to control Agro on plant selection/regeneration medium during transformation. The strategy is to use necessary levansucrase genes to disrupt the cysteine biosynthesis gene CysK in Agro to make the strain cysteine auxotrophic and also suicidal on plant tissue culture medium, which contains sucrose, at the same time. This concept works with other amino acid biosynthesis genes or other genes can accumulate toxic compounds on plant culture medium. The expected results would be no Agro overgrowth issue and consequently higher transformation efficiency and with no resources needed to clean up the Agro.

In one aspect, the instant disclosure provides a modified bacterial cell having a heterologous polynucleotide which has a nucleic acid sequence encoding a levansucrase enzyme. The levansucrase encoding sequence can be a SacB/SacR or SacB gene, which is inserted into the endogenous CysK gene such that the cell expresses the levansucrase enzyme, and has reduced endogenous CysK expression. In another aspect, the instant disclosure provides a population of the modified bacterial cells and methods of transforming a plant with the population of modified bacterial cells.

In another aspect, the instant disclosure further provides a polynucleotide having: from the 5′ to 3′: a 5′ fragment of a CysK coding sequence, a levansucrase coding sequence, and a 3′ fragment of the CysK coding sequence. The levansucrase coding sequence is a SacB/SacR gene or a SacB gene. In yet another aspect, the instant disclosure provides a method of transforming a cell including the steps of: (a) introducing the polynucleotide provided herein into a cell, culturing the cell, then selecting for the cells which express levansucrase and have reduced expression of CysK. In yet another embodiment, the instant disclosure provides a method of transforming a plant or plant part using the cell or the population of cells described herein, and the transformation efficiency is increased when compared to a transforming a plant or plant part with a control cell.

### III. Polynucleotides Comprising a Levansucrase Coding Sequence

The present disclosure provides polynucleotides containing a levansucrase coding sequence. In various embodiments, the polynucleotide containing from the 5′ to 3′: (a) a 5′ fragment of a CysK coding sequence; (b) a levansucrase coding sequence; and (c) a 3′ fragment of the CysK coding sequence. The levansucrase coding sequence can be a SacB/SacR gene, for example, a SacB/SacR gene isolated from Bacillus subtilis (NCBI accession: NC_000964.3: 3536012-3537433), or in other embodiments, the SacB/SacR gene is synthesized from the endogenous SacB/SacR gene isolated from Bacillus subtilis. In some embodiments, the SacB/SacR gene sequence shares at least 40%, at least 50%, at least 60%, at least 70%, at least 80%, at least 85%, at least 90%, at least 95%, or at least 99% sequence identity with SEQ ID NO: 2, and the corresponding levansucrase still retains the enzyme activity, that is, catalyzing the conversion of sucrose to levan. In some specific embodiments, the SacB/SacR gene consists of the nucleic acid sequence of SEQ ID NO: 2. The sequence of the SacB/SacR gene can be obtained from various sources. For example, from Bacillus subtilis genome sequence (NCBI accession: NC_000964.3: 3536012-3537433), or from GenBank: AB183144.1 (SEQ ID NO: 2).

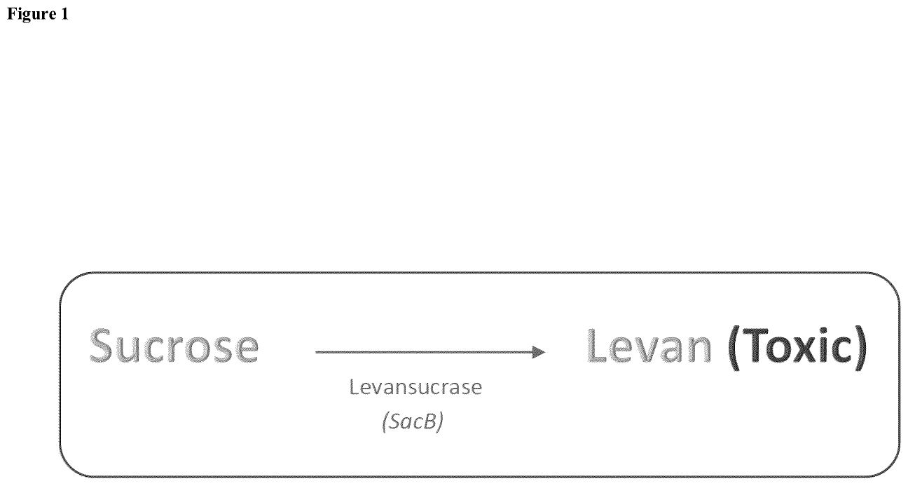

The SacB/SacR gene encodes a levansucrase (EC 2.4.1.10), which is an enzyme that catalyze the conversion of sucrose into levan (FIG. 1). Accordingly, levansucrase activity is the ability of a protein to convert sucrose into levan. Levan is a naturally occurring β-2,6-linked fructans with variable degrees of β-2,1-linked side chains present in many plants and microorganism species (Meyer, Advances in Food and Nutrition Research, 2015, 74: 47-91). Bacterial production of levan involves levansucrases and uses sucrose as the substrate. Levan cannot be metabolized by most Gram-negative bacteria, i.e., Agrobacterium tumefaciens and is therefore toxic to this group of microorganisms. Therefore, levan producing enzyme, i.e., levansucrase, can be used as a negative selection marker to suppress Agrobacterium overgrowth in Agrobacterium-mediated plant transformation (Liu et al., Front. Mol. Biosci., 2016, 3:70; doi: 10.3389/fmolb.2016.00070).

Levansucrase is isolated from levan-producing microbial strains and used in both free or immobilized forms for the synthesis of levan. In some embodiments, the levansucrase is a Bacillus subtilis levansucrase encoded by SacB/SacR. In some embodiments, the levansucrase shares at least 40%, at least 50%, at least 60%, at least 70%, at least 80%, at least 85%, at least 90%, at least 95%, or at least 99% sequence identity with SEQ ID NO: 1, and still retains the enzyme activity, that is, catalyzing the conversion of sucrose into levan. In some specific embodiments, the levansucrase consists of the sequence of SEQ ID NO: 1.

The polynucleotide further contains a promoter operably linked to the SacB/SacR gene. A number of promoters may be used in the practice of the present disclosure, one of ordinary skill in the art can select the promoter based on the desired outcome. The SacB/SacR gene can be operably linked to a constitutive, inducible, or other promoters for expression in a host cell. For example, a constitutive promoter can be selected from the list of, without limitations, T7AG, SV40, CMV, UBC, EF1A, PGK, ACTB, EF1α, PGK, UbC and CAGG promoters (Norman et al., PLoS ONE, 2010, 5(8): e12413; Qin et al., PLoS ONE, 2010, 5(5): e10611). In some embodiments, it can be a viral promoter such as endogenous promoters from the virus (e.g., the LTR of a lentiviral vector). In a preferred embodiment, the SacB/SacR gene is operably linked to a promoter that drives gene expression preferentially in the target cell. In some examples, heterologous polypeptide coding sequence is operably linked to a synthetic promoter, such as a JeT promoter (U.S. Pat. No. 6,555,674). In a specific embodiment, the promoter is a native promoter of SacB/SacR gene, which can be isolated from Genebank accession AB183144.

The polynucleotide can further include a RecA gene. RecA, also known as ECK2694, lexB, recH, rnmB, srf, tif, umuB or zab. In various embodiments, the RecA gene enclosed in the SacB/SacR recombination vector is from Escherichia coli, i.e., E. coli strain K12. In some embodiments, the sequence of RecA gene can derive from E. coli JE86-ST05 DNA sequence (Genebank accession: AP022815, from 3,244,037 to 3,245,098). In some embodiments, the RecA gene shares at least 40%, at least 50%, at least 60%, at least 70%, at least 80%, at least 85%, at least 90%, at least 95%, or at least 99% sequence identity with SEQ ID NO: 5. In a specific embodiment, the RecA gene consists of the sequence of SEQ ID NO: 5.

The RecA gene encodes a RecA protein, which is required for homologous recombination and the bypass of mutagenic DNA lesions by the SOS response. RecA catalyzes ATP-driven homologous pairing and strand exchange of DNA molecules necessary for DNA recombinational repair. RecA also catalyzes the hydrolysis of ATP in the presence of single-stranded DNA, the ATP-dependent uptake of single-stranded DNA by duplex DNA, and the ATP-dependent hybridization of homologous single-stranded DNAs. Therefore, the RecA protein can facilitate the homologous recombination of the polynucleotide containing a levansucrase-encoding sequence with at least one genomic DNA at, for example, the endogenous CysK coding sequence or a TetA gene loci of a host cell.

In some embodiments, the RecA protein is Escherichia coli RecA (GeneBank Accession: BCA75128.1), i.e., E. coli strain K12. In some embodiments, the RecA protein shares at least 40%, at least 50%, at least 60%, at least 70%, at least 80%, at least 85%, at least 90%, at least 95%, or at least 99% sequence identity with SEQ ID NO: 6. In a specific embodiment, the RecA protein consists of the sequence of SEQ ID NO: 6.

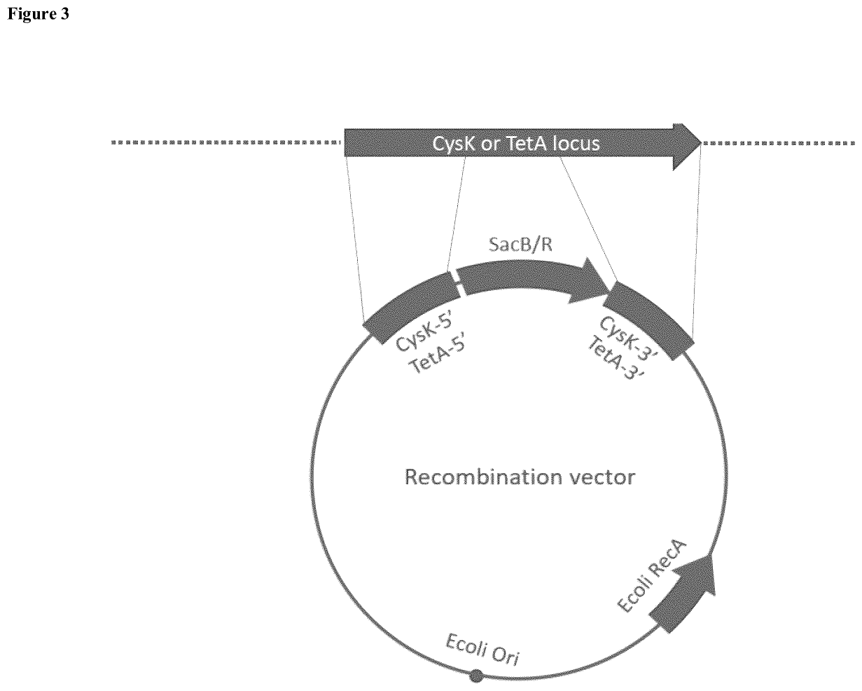

The polynucleotide further contains sequences flanking the 5′ or 3′ end of the levansucrase coding sequence, i.e., a SacB/SacR gene, and are complementary to at least part of an endogenous gene locus of a host cell (FIG. 3). For example, the sequence flanking the 5′ end of the SacB/SacR gene is complementary to a portion of the endogenous CysK coding sequence or TetA gene of a host; the sequence flanking the 3′ end of the SacB/SacR gene is complementary to a different portion of the endogenous CysK or TetA gene of a host. The portion complementary to the 5′ flanking sequence is upstream in respect to the portion complementary to the 3′ flanking sequence. In some embodiments, the length of the sequence flanking the 5′ end of the SacB/SacR gene and complementary to an endogenous gene can range from about 38, 39, 40, 50, 60, 70, 80, 90, 100, 110, 120, 130, 140, 150, 160, 170, 180, 190, 200, 300, 400, 500, 600, 700, 800, 900, or about 1000 or more base pairs. In some embodiments, the length of the sequence flanking the 3′ end of the SacB/SacR gene and complementary to an endogenous gene can range from about 38, 39, 40, 50, 60, 70, 80, 90, 100, 110, 120, 130, 140, 150, 160, 170, 180, 190, 200, 300, 400, 500, 600, 700, 800, 900, about 1000 or more base pairs.

The sequences flanking the 5′ or 3′ end of the levansucrase encoding sequence is sufficiently complementary to at least a portion of an endogenous gene such that homologous recombination occurs between the recombination vector and the part of genome in a host cell encoding the endogenous gene. In some embodiments, the 5′ flanking sequence is at least 60%, 65%, 70%, 75%, 80%, 85%, 90%, 95%, or 99% complementary to at least a portion of an endogenous gene, i.e., CysK gene or TetA gene, of a host cell. In a specific embodiment, the 5′ flanking sequence is completely complementary to at least a portion of an endogenous gene, i.e., CysK gene or TetA gene, of a host cell. In some embodiments, the 3′ flanking sequence is at least 60%, 65%, 70%, 75%, 80%, 85%, 90%, 95%, or 99% complementary to at least a portion of an endogenous gene, i.e., CysK gene or TetA gene, of a host cell. In a specific embodiment, the 3′ flanking sequence is completely complementary to at least a portion of an endogenous gene, i.e., CysK gene or TetA gene, of a host cell.

The flanking sequences complementary to at least a portion of an endogenous gene mediate homologous recombination of the polynucleotide with the endogenous gene. As a result, the SacB/SacR gene can be inserted into any locus within the endogenous CysK or TetA coding sequence. For example, the SacB/SacR gene can be inserted after the 1^{st}, the 2^{nd}, the 3^{rd}, the 4^{th}, the 5^{th}, the 6^{th}, the 7^{th}, the 8^{th}, the 9^{th}, the 10^{th}, . . . , or the 1037^{th} base pair of the endogenous CysK coding sequence. In addition, the homologous recombination further results in the inactivation of the endogenous gene of a host cell. In some embodiments, the homologous recombination results in the insertion of a SacB/SacR gene into the endogenous CysK locus and the inactivation of the endogenous CysK gene. In other embodiments, the homologous recombination results in the insertion of SacB/SacR gene into the endogenous TetA locus and the inactivation of the endogenous TetA gene. In some embodiments, the homologous recombination results in the reduced expression of the endogenous gene, i.e., the reduced expression of endogenous CysK gene. In other embodiments, the homologous recombination results in the reduced expression of the endogenous TetA gene. In various embodiments, the expression of endogenous CysK gene is reduced by at least 5%, 10%, 20%, 30%, 40%, 50%, 60%, 70%, 80%, 90%, 95%, 99%, or 100%. In other embodiments, the expression of endogenous TetA gene is reduced by at least 5%, 10%, 20%, 30%, 40%, 50%, 60%, 70%, 80%, 90%, 95%, 99%, or 100%.

The polynucleotide may further include additional elements. The polynucleotide may further include one or more than one T-DNA sequences. For example, a recombinant DNA construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a guide RNA of the present disclosure into nuclear DNA genome of a host plant (e.g., a pea plant).

The polynucleotide may further include additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In some preferred embodiments, the polynucleotide is a recombination vector. One with ordinary skills in the art can choose any suitable recombination known in the art, such as pUC plasmids. In one specific embodiment, the vector is pLVC18 (FIG. 3).

### IV. Modified Bacterial Cell

The present disclosure further provides a modified bacterial cell having a heterologous polynucleotide which contains a levansucrase-encoding polynucleotide. In various embodiments, the levansucrase-encoding polynucleotide is SacB/SacR gene. In some embodiments, the SacB/SacR gene is isolated from Bacillus subtilis (NCBI accession: NC_000964.3: 3536012-3537433), or in other embodiments, the SacB/SacR gene is synthesized from the endogenous SacB/SacR gene isolated from Bacillus subtilis. In some embodiments, the SacB/SacR gene shares at least 40%, at least 50%, at least 60%, at least 70%, at least 80%, at least 85%, at least 90%, at least 95%, or at least 99% sequence identity with SEQ ID NO: 2, and the encoded protein product still retains the enzyme activity, that is, catalyzing the conversion of sucrose into levan. In some specific embodiments, the SacB/SacR gene consists of the nucleic acid sequence of SEQ ID NO: 2.

The polynucleotide can be introduced into the bacterial cell using a variety of means known in the art. For example, the polynucleotide can be introduced via chemical transformation. Bacterial cells are mixed with the polynucleotide and briefly exposed to an elevated temperature, a process known as heat shock. First, bacterial cells are incubated with the polynucleotide on ice for 5-30 minutes in a polypropylene tube. Polystyrene tubes should be avoided, as DNA can adhere to the surface, reducing transformation efficiency. Traditionally, 17×100 mm round-bottom tubes have been used for best results. Using 1.5 mL microcentrifuge tubes may result in poor heat distribution due to smaller surface-to-volume ratios of cell suspension, which can reduce transformation efficiency by as much as 60-90%, especially for the higher-efficiency cells. Heat shock is performed at 37-42° C. for 25-45 seconds as appropriate. For smaller volumes of cells in smaller tubes, the heat-shock interval, which depends on the surface-to-volume ratio of the cell suspension, should be reduced. Heat-shocked cells are then returned to ice for ≥2 minutes before the next step.

The polynucleotide can also be introduced into the bacterial cell via electroporation. Electroporation involves using an electroporator to expose competent cells and DNA to a brief pulse of a high-voltage electric field. This treatment is believed to induce transient pores in cell membranes, which permit DNA entry into the cells. The most common type of electric pulse in bacterial transformation is exponential decay, where a set voltage is applied and allowed to decay over a few milliseconds, called the time constant. The applied voltage is determined by field strength (V/cm), where V is the initial peak voltage and cm is the measurement of the gap between the electrodes of the cuvette used. Typically, electroporation of bacteria utilizes 0.1 cm cuvettes (20-80 μL volume) and requires a field strength of >15 kV/cm.

The modified bacterial cell expresses a levansucrase. In some embodiments, the levansucrase is a Bacillus subtilis levansucrase encoded by SacB. In some embodiments, the levansucrase shares at least 40%, at least 50%, at least 60%, at least 70%, at least 80%, at least 85%, at least 90%, at least 95%, or at least 99% sequence identity with SEQ ID NO: 1, while still retaining the enzyme activity, that is, catalyzing the conversion of sucrose into levan. In some specific embodiments, the levansucrase consists of the sequence of SEQ ID NO: 1.

The levansucrase-encoding polynucleotide, after being introduced into the bacterial cell, may be inserted into the genomic DNA of the cell. For example, the levansucrase coding polynucleotide, i.e., a SacB/SacR gene, can be inserted into the endogenous CysK coding sequence of the cell. As a result, the expression of the endogenous CysK gene is reduced. In some embodiments, the endogenous CysK gene expression is reduced by at least 5%, 10%, 20%, 30%, 40%, 50%, 60%, 70%, 80%, 90%, 95%, 99%, or 100%. In some specific embodiments, the SacB/SacR gene is inserted after the 1^{st}, the 2^{nd}, the 3^{rd}, the 4^{th}, the 5^{th}, the 6^{th}, the 7^{th}, the 8^{th}, the 9^{th}, the 10^{th}, . . . , or the 1037^{th} base pair of the endogenous CysK coding sequence. In one specific embodiments, the mutation is mediated by homologous recombination between, i.e., the polynucleotide described herein, and the genomic DNA of a host cell at, i.e., the locus of the endogenous CysK coding sequence.

In some embodiments, the endogenous CysK gene shares at least 40%, at least 50%, at least 60%, at least 70%, at least 80%, at least 85%, at least 90%, at least 95%, or at least 99% sequence identity with any one of SEQ ID NO: 3, SEQ ID NO: 7 or SEQ ID NO: 9, and the encoded protein product still retains the enzyme activity. In some specific embodiments, the endogenous CysK gene sequence consists of SEQ ID NO: 3, SEQ ID NO: 7 or SEQ ID NO: 9. The endogenous CysK gene encodes CysK which is a cysteine synthase. Cysteine synthase catalyzes the last reaction of 1-cysteine synthesis in bacteria (Ma et al., Biotechnol Appl Biochem. 2019; 66(1):74-81). In some embodiments, the CysK shares at least 40%, at least 50%, at least 60%, at least 70%, at least 80%, at least 85%, at least 90%, at least 95%, or at least 99% sequence identity with any one of SEQ ID NO: 4, SEQ ID NO: 8 or SEQ ID NO: 10, while still retaining the enzyme activity. In one specific embodiment, the sequence of CysK consists of SEQ ID NO: 4, SEQ ID NO: 8 or SEQ ID NO: 10.

As a result of reduced CysK expression due to the genetic editing, the modified bacterial cell is cysteine auxotrophic. Therefore, the growth of the modified bacterial cell in medium or culture deficient in cysteine is inhibited. In various embodiments, the growth of modified bacterial cell is inhibited by at least 10%, 20%, 30%, 40%, 50%, 60%, 70%, 80%, 90%, 95%, 99%, or 100%. Furthermore, in various embodiments, because the modified bacterial cell expresses a levansucrase, the growth of the modified bacterial cell on a medium or culture containing sucrose is inhibited. In some embodiments, the growth of modified bacterial cell is inhibited by at least 10%, 20%, 30%, 40%, 50%, 60%, 70%, 80%, 90%, 95%, 99%, or 100%. In various embodiments, the growth of the modified bacterial cell on a medium or a culture both deficient in cysteine and containing sucrose is inhibited. In various embodiments, the growth of modified bacterial cell is inhibited by at least 10%, 20%, 30%, 40%, 50%, 60%, 70%, 80%, 90%, 95%, 99%, or 100%. In various embodiments, the medium or culture is a plant tissue culture.

In various embodiments, the growth of the modified bacterial cell on a medium or culture both deficient in cysteine and containing sucrose is inhibited to a degree greater than the same on a medium or culture deficient in cysteine but does not contain sucrose. In other embodiments, growth of the modified bacterial cell on a medium or culture both deficient in cysteine and containing sucrose is inhibited to a degree greater than the same on a medium or culture containing sucrose but not deficient in cysteine. In some embodiments, the modified bacterial cell on a medium or culture both deficient in cysteine and containing sucrose is inhibited, whereas the growth of the same on a medium or culture deficient in cysteine but does not contain sucrose is not. In other embodiments, the growth of the modified bacterial cell on a medium or culture both deficient in cysteine and containing sucrose is inhibited, whereas the growth of the same on a medium or culture containing sucrose but not deficient in cysteine is not. In various embodiments, the medium or culture is a plant tissue culture.

In various embodiments, the modified bacterial cell on a medium or culture both deficient in cysteine and containing sucrose is inhibited to a degree greater than a control cell on the same medium or culture. A “control cell” used herein is a counterpart bacterial cell without the two-action selection mechanism. For example, in some embodiments, a control cell can be a wild-type bacterial cell. In other embodiments, a control cell expresses a levansucrase but have wild-type endogenous CysK gene such that the CysK expression is not reduced. In another embodiment, the control cell has reduced CysK expression but does not express a levansucrase. In various embodiments, the growth of the modified bacterial cell is inhibited on a medium or culture both deficient in cysteine and containing sucrose while the growth of a control cell on the same medium or culture is not. In various embodiments, the medium or culture is a plant tissue culture.

In various embodiments, the modified bacterial cell is a Gram-negative bacterium. In some embodiments, the modified bacterial cell is an Agrobacterium. In some embodiments, the modified bacterial cell is Agrobacterium agile, Agrobacterium albertimagni, Agrobacterium aurantiacum, Agrobacterium larrymoorei, Agrobacterium radiobacter, Agrobacterium rhizogenes, Agrobacterium rubi, Agrobacterium tumefaciens, Agrobacterium vitis, or Agrobacterium sp. In preferred embodiments, the Agrobacterium is Agrobacterium tumefaciens.

Further provided herein is a population of modified bacterial cells. In some embodiments, the population of modified bacterial cells are heterogeneous. In some embodiments, about 10%, about 20%, about 30%, about 40%, about 50%, about 60%, about 70%, about 80%, about 90%, about 95%, about 99%, or about 100% cells of the population contain the introduced polynucleotide. In some specific embodiments, the about 75%, about 80%, about 85%, about 90%, about 95%, about 99%, or about 100% cells of the population contain the introduced polynucleotide. In some embodiments, about 10%, about 20%, about 30%, about 40%, about 50%, about 60%, about 70%, about 80%, about 90%, about 95%, about 99%, or 100% cells of the population express a levansucrase and have reduced expression of endogenous cysK gene. In some specific embodiments, about 75%, about 80%, about 85%, about 90%, about 95%, about 99%, or about 100% cells of the population express a levansucrase and have reduced expression of endogenous cysK gene. In some embodiments, the population of modified bacterial cells are homogenous. In some embodiments, the population has reduced level or activity of CysK and/or increased level or activity of levansucrase when compared to a population of control cells.

In other embodiments, the population of modified bacterial cells are homogenous. In some embodiments, the population of modified bacterial cells belong to the same lineage which is derived from a single cell.

### V. Method of Transforming a Cell

A modified cell can be generated by transforming the cell with the polynucleotide as described herein. The method of transforming a cell includes the steps of: (a) introducing the polynucleotide into a cell (b) culturing the cell; (c) selecting for the cells from which express levansucrase and have reduced expression of CysK.

As described above, the polynucleotide can be introduced into the bacterial cell using a variety of means known in the art. For example, the polynucleotide can be introduced via chemical transformation. Bacterial cells are mixed with the polynucleotide and briefly exposed to an elevated temperature, a process known as heat shock. First, bacterial cells are incubated with the polynucleotide on ice for 5-30 minutes in a polypropylene tube. Polystyrene tubes should be avoided, as DNA can adhere to the surface, reducing transformation efficiency. Traditionally, 17×100 mm round-bottom tubes have been used for best results. Using 1.5 mL microcentrifuge tubes may result in poor heat distribution due to smaller surface-to-volume ratios of cell suspension, which can reduce transformation efficiency by as much as 60-90%, especially for the higher-efficiency cells. Heat shock is performed at 37-42° C. for 25-45 seconds as appropriate. For smaller volumes of cells in smaller tubes, the heat-shock interval, which depends on the surface-to-volume ratio of the cell suspension, should be reduced. Heat-shocked cells are then returned to ice for ≥2 minutes before the next step.

The polynucleotide can also be introduced into the bacterial cell via electroporation. Electroporation involves using an electroporator to expose competent cells and DNA to a brief pulse of a high-voltage electric field. This treatment is believed to induce transient pores in cell membranes, which permit DNA entry into the cells. The most common type of electric pulse in bacterial transformation is exponential decay, where a set voltage is applied and allowed to decay over a few milliseconds, called the time constant. The applied voltage is determined by field strength (V/cm), where V is the initial peak voltage and cm is the measurement of the gap between the electrodes of the cuvette used. Typically, electroporation of bacteria utilizes 0.1 cm cuvettes (20-80 μL volume) and requires a field strength of >15 kV/cm.

After being introduced to the cell, the polynucleotide may engage in homologous recombination with the endogenous CysK coding sequence. In some embodiments, the homologous recombination introduces a mutation to the endogenous CysK gene of the cell. For example, the homologous recombination may introduce an insertion to the endogenous CysK coding sequence of the cell. In some embodiments, the insertion is an insertion of a levansucrase coding sequence, for example, a SacB-SacR gene. In some specific embodiments, the SacB-SacR gene is inserted after the 1^{st}, the 2^{nd}, the 3^{rd}, the 4^{th}, the 5^{th}, the 6^{th}, the 7^{th}, the 8^{th}, the 9^{th}, the 10^{th}, . . . , or the 1037^{th} base pair of the endogenous CysK coding sequence. In some embodiments, the insertion of the SacB-SacR gene results in the expression of levansucrase by the cell. In various embodiments, the expression of the endogenous CysK gene is reduced due to the insertion of the SacB-SacR gene. In some specific embodiments, the expression of the endogenous CysK gene is reduced by at least 5%, 10%, 20%, 30%, 40%, 50%, 60%, 70%, 80%, 90%, 95%, 99%, or 100%.

After the polynucleotide is introduced into the cell is cultured in accordance with a proper method known in the field for a duration. In some embodiments, a duration is about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 30 hours, about 36 hours, about 42 hours, about 48 hours, about 3 days, about 4 days, about 5 days, about 6 days, or about 7 days. One of ordinary skill in the art can determine the duration for culture based on specific conditions.

Various methods known in the art can be used to determine the expression of levansucrase and CysK. In some embodiments, the detection can be completed using blotting assays, including Western blots, Northern blots, and Southern blots. Such blotting assays are commonly used techniques in biological research for the identification and quantification of biological samples. These assays include first separating the sample components in gels by electrophoresis, followed by transfer of the electrophoretically separated components from the gels to transfer membranes that are made of materials such as nitrocellulose, polyvinylidene fluoride (PVDF), or Nylon. Analytes can also be directly spotted on these supports or directed to specific regions on the supports by applying vacuum, capillary action, or pressure, without prior separation. The transfer membranes are then commonly subjected to a post-transfer treatment to enhance the ability of the analytes to be distinguished from each other and detected, either visually or by automated readers.

In some other embodiments, the detection can be completed using an ELISA assay, which uses a solid-phase enzyme immunoassay to detect the presence of a substance, usually an antigen, in a liquid sample or wet sample. Antigens from the sample are attached to a surface of a plate. Then, a further specific antibody is applied over the surface so it can bind to the antigen. This antibody is linked to an enzyme, and, in the final step, a substance containing the enzyme's substrate is added. The subsequent reaction produces a detectable signal, most commonly a color change in the substrate.

In various embodiments, the cell is a Gram-negative bacterium. In some embodiments, the bacterial cell is an Agrobacterium. In some embodiments, the bacterial cell is Agrobacterium agile, Agrobacterium albertimagni, Agrobacterium aurantiacum, Agrobacterium larrymoorei, Agrobacterium radiobacter, Agrobacterium rhizogenes, Agrobacterium rubi, Agrobacterium tumefaciens, Agrobacterium vitis, or Agrobacterium sp. In preferred embodiments, the Agrobacterium is Agrobacterium tumefaciens. In some specific embodiments, the cell is a competent bacterial cell. In one specific embodiment, the bacterial is AGL1 competent Agrobacterium.

As a result of reduced CysK expression due to the genetic editing, the modified bacterial cell is cysteine auxotrophic. Therefore, the growth of the modified bacterial cell in medium or culture deficient in cysteine is inhibited. In various embodiments, the growth of modified bacterial cell is inhibited by at least 10%, 20%, 30%, 40%, 50%, 60%, 70%, 80%, 90%, 95%, 99%, or 100%. Furthermore, in various embodiments, because the modified bacterial cell expresses a levansucrase, the growth of the modified bacterial cell on a medium or culture containing sucrose is inhibited. In some embodiments, the growth of modified bacterial cell is inhibited by at least 10%, 20%, 30%, 40%, 50%, 60%, 70%, 80%, 90%, 95%, 99%, or 100%. In various embodiments, the growth of the modified bacterial cell on a medium or a culture both deficient in cysteine and containing sucrose is inhibited. In various embodiments, the growth of modified bacterial cell is inhibited by at least 10%, 20%, 30%, 40%, 50%, 60%, 70%, 80%, 90%, 95%, 99%, or 100%. In various embodiments, the medium or culture is a plant tissue culture.

### VI. Method of Transforming a Plant Using the Modified Bacterial Cell

In another aspect, the instant disclosure provides a method of transforming a plant or a plant part (e.g., protoplast, juice, pulp, seed, fruit, flower, nectar, embryo, pollen, ovule, leaf, stem, branch, bark, kernel, ear, cob, husk, stalk, root, root tip, anther) using the modified bacterial cells or a population thereof, by introducing a polynucleotide of interest into the plant or a plant part. Also disclosed herein are methods of transforming a plant or plant part by introducing into the plant or the plant part a polynucleotide of interest and regenerating a transformed plant or plant part from said plant cell. The transformation efficiency is increased when compared to transforming a plant or plant part with a control cell.

The polynucleotide of interest can comprise a variety of genes which confers a trait, e.g., an advantageous agronomic trait. Accordingly, the polynucleotide of interest can be various insect resistance genes which are operably linked to the promoters and/or included in the DNA construct disclosed herein. As examples of insect resistance genes that can be operably linked to the regulatory elements of the subject disclosure, the following traits are provided. Genes that provide exemplary Lepidopteran insect resistance include: cry1A; cry1A.105; cry1Ab; cry1Ab (truncated); cry1Ab Ac (fusion protein); cry1Ac; cry1C; cry1F; cry1Fa2; cry2Ab2; cry2Ae; cry9C; mocry1F; pinII (protease inhibitor protein); vip3A(a); and vip3Aa20. Genes that provide exemplary Coleopteran insect resistance include: cry34Ab1; cry35Ab1; cry3A; cry3Bb1; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

Various herbicide tolerance genes can be operably linked to the promoters and/or included in the DNA construct disclosed herein. As examples of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to any promoters disclosed herein. For example, selectable marker genes include, but are not limited to genes encoding glyphosate resistance genes such as: mutant EPSPS genes including 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). Resistance genes for glufosinate and/or bialaphos compounds include dsm-2, bar and pat genes. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Acc1-S1, Acc1-S2 and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1s+ genes (tolerance to triazine), and nitrilase genes (tolerance to benzonitrile). The above list of herbicide tolerance genes is not meant to be limiting. Any herbicide tolerance genes are encompassed by the present disclosure.

Various agronomic trait genes can be operably linked to the promoters and/or included in the DNA construct disclosed herein. As examples of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

Various selectable markers also described as reporter genes can be operably linked to the promoters and/or included in the DNA construct disclosed herein. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes are utilized for selection of transformed cells or tissues. Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Selectable marker genes include genes encoding antibiotic resistance, such as those encoding neomycin phosphotransferase II (NEO), spectinomycin/streptinomycin resistance (AAD or SpcR), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

Herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Acc1-S1, Acc1-S2 and Acc1-S3. Herbicides can also inhibit photosynthesis, including triazine (psbA and 1s+ genes) or benzonitrile (nitrilase gene). Further, such selectable markers can include positive selection markers such as phosphomannose isomerase (PMI) enzyme.

Selectable marker genes can further include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). Selectable marker genes can further include genes encoding resistance to: chloramphenicol;

methotrexate; hygromycin; spectinomycin; bromoxynil; glyphosate; and phosphinothricin.

Other selectable marker genes that could be employed on the expression constructs disclosed herein include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414), red fluorescent protein (DsRFP, RFP, etc), beta-galactosidase, and the maize genes encoding for anthocyanin production (Ludwig, et al., (1990) Science 247:449), and the like (See Sambrook, et al., Molecular Cloning: A Laboratory Manual, Third Edition, Cold Spring Harbor Press, N.Y., 2001), herein incorporated by reference in their entirety. The above list of selectable marker genes is not meant to be limiting. Any reporter or selectable marker gene are encompassed by the present disclosure.

The polynucleotides of interest can be synthesized for optimal expression in a plant. For example, a polynucleotide of interest can have been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. For example, a polynucleotide of interest, e.g., a coding sequence, gene, heterologous coding sequence, or transgene/heterologous coding sequence can be designed to be expressed in plants at a higher level resulting in higher transformation efficiency. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

The expression levels of polynucleotides of interest can be measured by any methods known in the art. For example, polynucleotide expression levels can be measured by quantifying levels of the polynucleotide product, e.g., an RNA or a protein, by, e.g., PCR, real-time PCR, Western blotting, and ELISA. Polynucleotide expression levels can also be assessed by quantifying levels of function of polynucleotide product, for example by quantifying the occurrence of events caused by the polynucleotide product (e.g., morphology and number of regenerated shoots) or by quantifying the levels of product produced by the polynucleotide product, as further disclosed elsewhere in the present disclosure.

The term “transform” or “transformation” refers to any method used to introduce polypeptides or polynucleotides into plant cells. For purpose of the present disclosure, the transformation can be: “stable transformation”, wherein the transformation construct [e.g., a construct including a polynucleotide of interest encoding an editing reagent (e.g., a nuclease, a guide RNA), a repair template, a morphogen, a transforming protein, a recombination modulator, a repair modulator, a defense pathway modulator, a selectable marker, a regulatory RNA, a small RNA, an enzyme, a transcription factor, a receptor, a ligand, a molecule that confers resistance to pests or disease, tolerance to herbicides, and/or advantageous agronomic traits, for use in the methods of the present invention] is introduced into a host (e.g., a host plant, plant part, plant cell, etc.) and integrates into the genome of the host and is capable of being inherited by the progeny thereof; or “transient transformation”, wherein the transformation construct is introduced into a host (e.g., a host plant, plant part, plant cell, etc.) and expressed temporarily. The methods disclosed herein can also be used for insertion of heterologous polynucleotides and/or modification of native plant gene expression to achieve desirable plant traits.

The promoters disclosed herein and/or any polynucleotide of interest operably linked to a promoter disclosed herein can be introduced into a plant cell, organelle, or plant embryo by a variety of means of transformation, including microinjection (Crossway et al. (1986) Biotechniques 4:320-334), electroporation (Riggs et al. (1986) Proc. Natl. Acad. Sci. USA 10 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration [see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens)]; all of which are herein incorporated by reference.

Agrobacterium- and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, viral infection, Agrobacterium and viral mediated (Caulimoriviruses, Geminiviruses, RNA plant viruses), liposome mediated and the like. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

More than one polynucleotides of interest, e.g., polynucleotides encoding an editing reagent (e.g., a nuclease, a guide RNA), a repair template, a morphogen, a transforming protein, a recombination modulator, a repair modulator, a defense pathway modulator, a selectable marker, a regulatory RNA, a small RNA, an enzyme, a transcription factor, a receptor, a ligand, a molecule that confers pest resistance, disease resistance, herbicide tolerance, and/or advantageous agronomic traits (e.g., yield improvement, nitrogen use efficiency, water use efficiency, and nutritional quality) can be introduced into the plant, plant cell, plant organelle, or plant embryo simultaneously or sequentially. More than one polynucleotides of interest can be introduced into the plant, plant cell, plant organelle, or plant embryo by introducing one DNA construct that comprise all the polynucleotides of interest operably linked to one or more promoters. Alternatively, more than one polynucleotides of interest can be introduced into the plant, plant cell, plant organelle, or plant embryo by introducing more than one DNA constructs that each comprise some of the polynucleotides of interest operably linked to one or more promoters simultaneously or sequentially. For example, editing reagents (e.g., a nuclease and a guide RNA), and a repair template can be introduced into the plant, plant cell, plant organelle, or plant embryo in one DNA construct, or in more than one DNA construct simultaneously or sequentially. The amount or ratio of more than one polynucleotides of interest, or molecules encoded therein, can be adjusted by adjusting the amount or concentration of the polynucleotides and/or timing and dosage of introducing the polynucleotides into the plant or plant part. In specific embodiments, polynucleotides of interest encode a nuclease, a guide RNA(s), and a repair template, and the ratio of the nuclease (or encoding nucleic acid) to the guide RNA(s) (or encoding DNA) generally will be about stoichiometric such that the two components can form an RNA-protein complex with the target DNA. The ratio of the nuclease (or encoding nucleic acid) and/or the guide RNA(s) (or encoding DNA) to the repair template will be optimized to facilitate HDR.

The cells that have been transformed may be cultured and grown into plants in accordance with conventional ways. See, for example, McCormick et al. (1986) Plant Cell Reports 5:81-84. In this manner, the present invention provides transformed plants or plant parts, transformed seed (also referred to as “transgenic seed”) or transformed plant progenies having a nucleic acid modification stably incorporated into their genome.

Also disclosed herein are plants and plant parts generated by the methods of the present disclosure, and plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant concentrate (e.g., whole plant concentrate or plant part concentrate), plant powder [e.g., formulated powder, such as formulated plant part powder (e.g., seed flour)], and plant biomass (e.g., dried biomass, such as crushed and/or powdered biomass), and food or beverage products obtained from plants of the present disclosure. Also provided herein are seeds, such as a representative sample of seeds, from a plant generated by the methods of the present disclosure.

A transformed plant cell, callus, tissue or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed plants and plant cells can also be identified by screening for the activities of any visible marker genes (e.g., the 3-glucuronidase, luciferase, or green fluorescent protein genes) that may be present on the recombinant nucleic acid constructs. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay is a method of detecting and quantifying the presence of a DNA sequence. Briefly, a FRET oligonucleotide probe is designed with one oligo within the transgene/heterologous coding sequence and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

A method of detecting and quantifying the presence of a DNA sequence by detecting an amplification reaction can be used. Briefly, the genomic DNA sample comprising the integrated gene expression cassette polynucleotide is screened using a polymerase chain reaction (PCR) based assay. The assay can utilize a PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction.

The confirmation methods include a long read NGS (Next-Generation Sequencing), which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The confirmation methods also include is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The confirmation methods also include a short read technology that uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

A NGS approach can use single DNA molecules for the sequence reactions, e.g., by producing up to 800 million short reads that result in 21 gigabases per run. These reactions are completed using fluorescent dye-labelled virtual terminator nucleotides that is described as a “sequencing by synthesis” approach. A NGS approach can also use a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

In another embodiment, the detection can be completed using blotting assays, including Western blots, Northern blots, and Southern blots. Such blotting assays are commonly used techniques in biological research for the identification and quantification of biological samples. These assays include first separating the sample components in gels by electrophoresis, followed by transfer of the electrophoretically separated components from the gels to transfer membranes that are made of materials such as nitrocellulose, polyvinylidene fluoride (PVDF), or Nylon. Analytes can also be directly spotted on these supports or directed to specific regions on the supports by applying vacuum, capillary action, or pressure, without prior separation. The transfer membranes are then commonly subjected to a post-transfer treatment to enhance the ability of the analytes to be distinguished from each other and detected, either visually or by automated readers.

In a further embodiment the detection can be completed using an ELISA assay, which uses a solid-phase enzyme immunoassay to detect the presence of a substance, usually an antigen, in a liquid sample or wet sample. Antigens from the sample are attached to a surface of a plate. Then, a further specific antibody is applied over the surface so it can bind to the antigen. This antibody is linked to an enzyme, and, in the final step, a substance containing the enzyme's substrate is added. The subsequent reaction produces a detectable signal, most commonly a color change in the substrate.

In some embodiments, the transformed plant is heterogeneous. A “heterogeneous” plant, as used herein, refers to the plant of which not all the cells have the same genotype. In some specific embodiments, the transformed plant is homogeneous. A “heterogeneous” plant, as used herein, refers to the plant of which all the cells have the same genotype. In some embodiments, a homogeneous transformed plant is developed from a single transformed plant cell.

In various embodiments, the cell is a Gram-negative bacterium. In some embodiments, the bacterial cell is an Agrobacterium. In some embodiments, the bacterial cell is Agrobacterium agile, Agrobacterium albertimagni, Agrobacterium aurantiacum, Agrobacterium larrymoorei, Agrobacterium radiobacter, Agrobacterium rhizo genes, Agrobacterium rubi, Agrobacterium tumefaciens, Agrobacterium vitis, or Agrobacterium sp. In preferred embodiments, the Agrobacterium is Agrobacterium tumefaciens.

As a result of reduced CysK expression due to the genetic editing, the modified bacterial cell is cysteine auxotrophic. Therefore, the growth of the modified bacterial cell in medium or culture deficient in cysteine is inhibited. In various embodiments, the growth of modified bacterial cell is inhibited by at least 10%, 20%, 30%, 40%, 50%, 60%, 70%, 80%, 90%, 95%, 99%, or 100%. Furthermore, in various embodiments, because the modified bacterial cell expresses a levansucrase, the growth of the modified bacterial cell on a medium or culture containing sucrose is inhibited. In some embodiments, the growth of modified bacterial cell is inhibited by at least 10%, 20%, 30%, 40%, 50%, 60%, 70%, 80%, 90%, 95%, 99%, or 100%. In various embodiments, the growth of the modified bacterial cell on a medium or a culture both deficient in cysteine and containing sucrose is inhibited. In various embodiments, the growth of modified bacterial cell is inhibited by at least 10%, 20%, 30%, 40%, 50%, 60%, 70%, 80%, 90%, 95%, 99%, or 100%. In various embodiments, the medium or culture is a plant tissue culture.

In various embodiments, the efficiency of transforming a plant or a plant part using the modified bacterial cell is increased when compared to transforming a plant or a plant part using a control cell. In some embodiments, a control cell can be a wild-type bacterial cell. In some embodiments, a control cell expresses a levansucrase but the endogenous CysK gene expression is not reduced. In another embodiment, the control cell has reduced CysK expression but does not express a levansucrase. In some embodiments, the efficiency of transforming a plant or a plant part is increased by at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000% compared to transforming a plant or a plant part using a control cell. In some embodiments, the transformation efficiency of a plant or a plant part is increased when compared to transforming a plant or plant part with a control cell because the growth of the modified bacterial cell is inhibited by both the presence of sucrose and the deficiency in cysteine.

The plant or plant part, which can be used for transformation herein can be any plant. In some embodiments, the plant is a monocot. The term “monocot” or “monocotyledons” as used herein refers to grass and grass-like flowering plants of which the seeds typically contain only one embryonic leaf, or cotyledon. In agriculture the majority of the biomass produced comes from monocotyledons. Commonly known monocot plants include, without limitations, major grains (rice, wheat, maize, etc.), forage grasses, sugar cane, bamboos, Arecaceae, bananas and Musaceae, gingers and their relatives, turmeric and cardamom, asparagus, pineapple, sedges, Juncaceae, leeks, onion and garlic, lilies, daffodils, irises, amaryllis, cannas, bluebells and tulips. In other embodiments, the plant is a dicot. The term “dicot” or “dicotyledons” refers to flowering plants of which the seeds typically contain two embryonic leaves or cotyledons. Most common garden plants, shrubs and trees, and broad-leafed flowering plants such as magnolias, roses, geraniums, and hollyhocks are dicots.

Commonly known plant species includes, without limitations, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of dicots, e.g., legumes.

### EXAMPLES

### Example 1

Current common practice was to use antibiotics such as Timentin to control Agro overgrowth. However, Agrobacterium overgrowth issues are common with 30-50% of experiments and loss of about 4% experiments due to Agro overgrowth. Moreover, the time cost for washing explants and to transferring them to fresh medium was extensive.

To overcome the challenge Agrobacterium overgrowth, the strategy was to use necessary levansucrase genes to disrupt the cysteine biosynthesis gene CysK in Agro to make the strain cysteine auxotrophic and also suicidal on plant tissue culture medium at the same time. The unique feature was that mutant strains of Agrobacterium was created with two Mode-of-Actions. This innovation could solve the Agro overgrowth issue completely. This concept works with other amino acid biosynthesis genes or other genes can accumulate toxic compounds on plant culture medium. The advantages of this strategy is the system has two mode-of-actions to control Agro on plant selection/regeneration medium during transformation. The expected results would be no Agro overgrowth issue and consequently higher transformation efficiency and with no resources needed to clean up the Agro. This technology was expected to solve these problems, such as washing explants and transferring them to fresh medium.

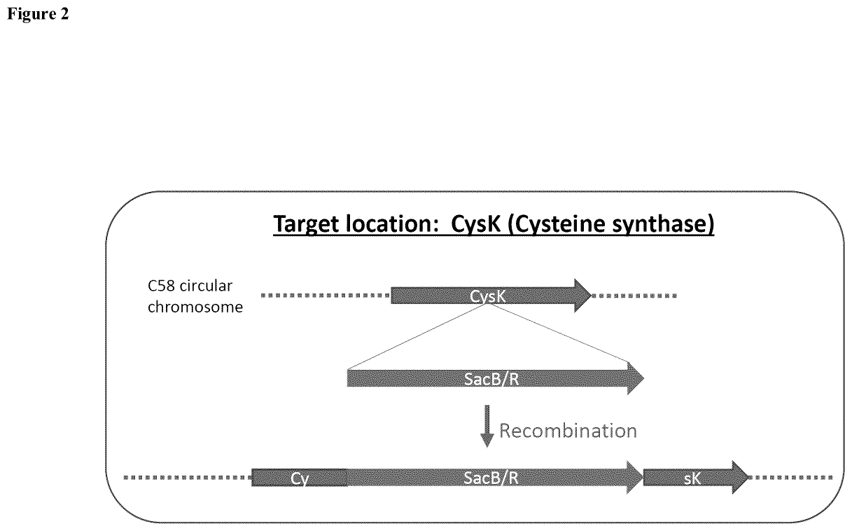

SacB (Levansucrase) and SacR (Levansucrase repressor) convert sucrose to levan, which is toxic to gram negative bacteria (FIG. 1). An example of the mutant strain with two Mode-of-Actions can be constructed by inserting and expressing SacB gene at Agrobacterium C58 CysK gene loci, such that the Agrobacterium C58 CysK gene is mutated (FIG. 2). The mutant strain cannot grow on plant tissue culture because sucrose will be convert to levan and kill the mutant strain, and the mutant strain needs cysteine for growth, which the tissue culture medium does not contain.

An example recombination vector which can be used in the process above is pLVC18, which contains part of a ′5 CysK coding sequence, followed by a SacB/SacR coding sequence, the followed by a 3′ CysK coding sequence (FIG. 3). This vector, upon transformation into Agrobacterium, will be engaged in homologous recombination with Agrobacterium genome at CysK loci.

1. A modified bacterial cell comprising a heterologous polynucleotide comprising a nucleic acid sequence encoding a levansucrase enzyme, wherein an endogenous CysK gene has reduced expression when compared to a proper control bacterium.

2. (canceled)

3. The modified bacterial cell of claim 1, wherein an endogenous CysK gene expression is reduced due to a mutation in the CysK gene or regulatory region of the CysK gene, wherein the mutation is an insertion.

4. (canceled)

5. The modified bacterial cell of claim 3, wherein the insertion is an insertion of a heterologous polynucleotide comprising a nucleic acid sequence encoding the levansucrase enzyme, wherein the nucleic acid molecule encoding the levansucrase enzyme is a SacB/SacR gene.

6. (canceled)

7. The modified bacterial cell of claim 5, wherein the SacB/SacR gene comprises
(i) at least 80%, or at least 85%, or at least 90%, or at least 95%, or at least 99% sequence identity with SEQ ID NO: 2, and retains levansucrase activity; or
(ii) the nucleotide sequence of SEQ ID NO: 2.

8. The modified bacterial cell of claim 1, wherein the levansucrase enzyme comprises
(i) at least 80%, or at least 85%, or at least 90%, or at least 95%, or at least 99% amino acid sequence identity with SEQ ID NO: 1, and retains levansucrase activity; or
(ii) the amino acid sequence of SEQ ID NO: 1.

9. The modified bacterial cell of claim 1, wherein the endogenous CysK coding sequence comprises
(i) at least 80%, or at least 85%, or at least 90%, or at least 95%, or at least 99% sequence identity with any one of SEQ ID NO: 3, SEQ ID NO: 7 or SEQ ID NO: 9, and retains CysK activity; or
(ii) the nucleotide sequence of SEQ ID NO: 3, SEQ ID NO: 7 or SEQ ID NO: 9; or wherein the endogenous CysK protein comprises
(iii) at least 80%, or at least 85%, or at least 90%, or at least 95%, or at least 99% sequence identity with any one of SEQ ID NO: 4, SEQ ID NO: 8 or SEQ ID NO: 10, and retains CysK activity; or
(iv) the amino acid sequence of SEQ ID NO: 4, SEQ ID NO: 8 or SEQ ID NO: 10.

10.-11. (canceled)

12. The modified bacterial cell of claim 1, wherein the modified bacterial cell is cysteine auxotrophic.

13. (canceled)

14. The modified bacterial cell of claim 1, wherein the modified bacterial cell is an Agrobacterium.

15. The modified bacterial cell of claim 1, wherein the modified bacterial cell further comprises one or more heterologous polynucleotide of interest.

16. The modified bacterial cell of claim 15, wherein the one or more heterologous polynucleotide of interest encodes an editing reagent and a repair template, and wherein homology-directed repair (HDR) is increased, an HDR to non-homologous end-joining (NHEJ) ratio is increased, chimerism is reduced, mosaicism is reduced, and/or uniform editing of a plant genome is increased relative to a control plant or plant part, when the DNA construct is introduced in a plant or plant part.

17. (canceled)

18. The modified bacterial cell of claim 1, wherein the growth of the modified bacterial cell in a plant tissue culture medium deficient in cysteine is inhibited; or
wherein the growth of the modified bacterial cell in a plant tissue culture medium comprising sucrose is inhibited.

19.-20. (canceled)

21. The modified bacterial cell of claim 1, wherein the growth of the modified bacterial cell in a plant tissue culture medium comprising sucrose and deficient in cysteine is inhibited when compared to a control cell.

22. (canceled)

23. A population of the modified bacterial cell of claim 1.

24.-28. (canceled)

29. A polynucleotide comprising from the 5′ to 3′:
(a) a 5′ fragment of a CysK coding sequence;
(b) a levansucrase coding sequence; and
(c) a 3′ fragment of the CysK coding sequence.

30. The polynucleotide of claim 29, wherein the levansucrase enzyme coding sequence is a SacB/SacR gene.

31. The polynucleotide of claim 30, wherein the SacB/SacR gene comprises
(i) at least 80%, or at least 85%, or at least 90%, or at least 95%, or at least 99% sequence identity with SEQ ID NO: 2, and retains levansucrase activity; or
(ii) the polynucleotide sequence of SEQ ID NO: 2.

32. The polynucleotide of claim 29, wherein the levansucrase comprises
(i) at least 80%, or at least 85%, or at least 90%, or at least 95%, or at least 99% sequence identity with SEQ ID NO: 1, and retains levansucrase activity; or
(ii) the amino acid sequence SEQ ID NO: 1.

33. The polynucleotide of claim 29, wherein the CysK coding sequence comprises
(i) at least 80%, or at least 85%, or at least 90%, or at least 95%, or at least 99% sequence identity with any one of SEQ ID NO: 3, SEQ ID NO: 7 or SEQ ID NO: 9, and retains CysK activity; or
(ii) the polynucleotide sequence of SEQ ID NO: 3, SEQ ID NO: 7 or SEQ ID NO: 9; or wherein the CysK protein comprises
(i) at least 80%, or at least 85%, or at least 90%, or at least 95%, or at least 99% sequence identity with any one of SEQ ID NO: 4, SEQ ID NO: 8 or SEQ ID NO: 10, and retains CysK activity; or
(ii) the amino acid sequence of SEQ ID NO: 4, SEQ ID NO: 8 or SEQ ID NO: 10.

34. (canceled)

35. The polynucleotide of claim 29, further comprising a RecA gene.

36.-37. (canceled)

38. A method of transforming a cell, comprising the steps of:
(a) introducing the polynucleotide of claim 29 into a cell;
(b) culturing the cell;
(c) selecting for the cells from step (b) which express levansucrase and have reduced expression of CysK.

39. The method of claim 38, wherein a SacB/SacR gene is inserted into the CysK coding sequence, wherein insertion of the SacB/SacR gene into the endogenous CysK coding sequence results the expression of the SacB/SacR gene and reduces the expression of the endogenous CysK gene.

40.-41. (canceled)

42. The method of claim 38, further comprising introducing at least one heterologous polynucleotide of interest into the cell in step (a).

43. The method of claim 42, wherein the at least one heterologous polynucleotide of interest encodes an editing reagent and a repair template, and wherein homology-directed repair (HDR) is increased, an HDR to non-homologous end-joining (NHEJ) ratio is increased, chimerism is reduced, mosaicism is reduced, and/or uniform editing of a plant genome is increased relative to a control plant or plant part, when the DNA construct is introduced in a plant or plant part.

44.-45. (canceled)

46. The method of claim 38, wherein the cell is an Agrobacterium.

47. A method of transforming a plant or plant part using the cell of claim 1, wherein the transformation efficiency is increased when compared to transforming a plant or plant part with a control cell, wherein the plant is an explant or seed; or
wherein the plant is a soybean plant.

48.-51. (canceled)
